(19) United States
(12) Patent Application Publication
(10) Pub. No.: US 2009/0163749 A1
(43) Pub. Date: Jun. 25, 2009

(54) CONVERSION OF METHANE INTO C3˜C13 HYDROCARBONS

(75) Inventors: Wensheng Li, Changsha, Hunan (CN); Li Huang, Changsha (CN); Yanqun Ren, Changsha (CN); Xiaoping Zhou, Changsha (CN)

Correspondence Address: BAKER & MCKENZIE LLP, Pennzoil Place, South Tower, 711 Louisiana, Suite 3400, HOUSTON, TX, 77002-2716, US

(73) Assignee: MICROVAST TECHNOLOGIES, LTD., Huzhou (CN)

(21) Appl. No.: 12/346,381

(22) Filed: Dec. 30, 2008

Related U.S. Application Data
(63) Continuation-in-part of application No. 12/293,663.

(30) Foreign Application Priority Data
Mar. 20, 2006 (CN) CN200610031377
Mar. 12, 2007 (CN) PCT/CN2007/000780

(51) Int. Cl. C07C 2/00

(52) U.S. Cl. 585/310

(57) ABSTRACT

A process for converting methane into C₃˜C₁₃ hydrocarbons is provided including the steps of reacting methane with oxygen and HBr/H₂O over a first catalyst in a first reactor to methane bromides and converting the methane bromides into C₃˜C₁₃ hydrocarbons and HBr over a second catalyst in a second reactor. The process may further include recovering HBr produced in the second reactor and recylcing it into the first reactor.

### FIELD OF THE INVENTION

This invention is a Continuation-In-Part (CIP) application of U.S. application Ser. No. 12293663. The present invention relates to a process for preparing C₃˜C₁₃ hydrocarbons from methane.

### FEDERALLY SPONSORED RESEARCH

Not applicable.

### REFERENCE TO MICROFICHE APPENDIX

Not applicable.

### BACKGROUND OF THE INVENTION

Natural gas is the most abundant hydrocarbon resource on earth besides coal, and is mainly composed of methane with a small amount of other compounds such as ethane, propane, steam, and carbon dioxide. Compared with coal, natural gas is a cleaner hydrocarbon resource because it can be directly used as fuel or chemical feedstock to produce other chemical products. Since most natural gas resources are often discovered in remote areas and natural gas is difficult to compress and transport, the cost to use natural gas is quite high. On the other hand, the high stability of C—H bonds of methane makes the chemical conversion difficult. In currently available technologies, natural gas is mostly used to make hydrogen or synthesis gas (H₂+CO) (also referred to as “syngas”) with the hydrogen being used to produce ammonia, and the syngas converted to methanol. Although the Fischer-Tropsch method can convert natural gas into fuel oil through a syngas process, the cost is higher than that of original petroleum refining method. Therefore, natural gas is not widely used as a substitute for petroleum to produce fuel oil or other chemical monomers. A new process for converting methane into easily transported liquid petroleum or other synthesis intermediates is thus desired. Since the syngas route is not a cost-effective process, it has been suggested to produce higher value chemicals from light alkanes by selective oxidation processes. Except for a few successful examples such as preparing maleic anhydride by oxidation of n-butane, most cases of selective oxidation method of light alkanes, such as CH₄, C₂H₆ and C₃H₈, did not achieve successful application in chemical industry because of low conversion rate, low selectivity, and difficulty to separate the products.

Another method involves converting methane into methanol [Roy A, Periana et al., Science, 280, 560 (1998)] and acetic acid [Roy A. Periana, et al., Science, 301, 814 (2003)]. In such process, SO₂ was produced that could not be recovered, and concentrated sulphuric acid, which was used as reactant and solvent, was diluted after the reaction and could not be used continuously. This method has not been industrialized.

In the earlier paper [G. A. Olah et al. Hydrocarbon Chemistry (Wiley, New York, 1995)], Olah reported the process to form CH₃Br and HBr by reacting methane and Br₂, then to hydrolyze CH₃Br to provide methanol and dimethyl ether. This report did not suggest or disclose how to recycle HBr. The object of such process was not to synthesize hydrocarbons, and the reported single-pass conversion rate of methane was lower than 20%. The inventors of the present invention had also designed a process to convert alkanes to methanol and dimethyl ether (Xiao Ping Zhou et al., Chem. Commun. 2294 (2003); Catalysis Today 98, 317 (2004); U.S. Pat. No. 6,486,368; U.S. Pat. No. 6,472,572; U.S. Pat. No. 6,465,696; U.S. Pat. No. 6,462,243). Such process, however, related to the use of Br₂ and the extra step of regenerating Br₂. As known, the utilization and storage of vast amount of Br₂ is very dangerous.

### SUMMARY OF THE INVENTION

Some embodiments of the present invention offer an efficient way to convert methane into higher hydrocarbons. One embodiment of the present invention provides an efficient way to convert methane of natural gas into liquid hydrocarbons or easily-liquified hydrocarbons. In certain embodiments, hydrogen bromide is used as a media in some embodiments of the invention to convert methane or natural gas into C₃˜ C₁₃ hydrocarbons.

In some embodiments of the invention, a process for preparing C₃˜C₁₃ hydrocarbons from methane, oxygen and HBr/H₂O is provided including the steps of reacting methane with oxygen and HBr/H₂O over a first catalyst in a first reactor to form CH₃Br and CH₂Br₂; converting CH₃Br and CH₂Br₂ into C₃˜C₁₃ hydrocarbons and HBr over a second catalyst in a second reactor; and recovering the HBr produced in the second reactor. The first catalyst and the second catalyst are also provided respectively.

### DETAILED DESCRIPTION OF EMBODIMENTS OF THE INVENTION

The following description illustrates embodiments of the invention by way of example and not by way of limitation. Thus, the embodiments described below represent preferred embodiments of the invention. All numbers disclosed herein are approximate values unless stated otherwise, regardless whether the word “about” or “approximately” is used in connection therewith. The numbers may vary by up to 1%, 5%, or sometimes 10% to 20%. Whenever a numerical range with a lower limit and an upper limit is disclosed, any number falling within the range is specifically and expressly disclosed.

The present invention provides a chemical process that enables methane and/or natural gas to be converted into higher molecular weight hydrocarbons, using hydrogen bromide to activate C—H bonds in the feedstock. In some embodiments of the present invention, methane is converted into one or more alkyl bromides which are then converted into higher hydrocarbons. Further, in some embodiments, the produced HBr can be collected and directed into the first reactor for re-use. As used herein, the term “higher hydrocarbons” refers to hydrocarbons having a greater number of carbon atoms than two.

Generally, the present process comprises the steps of:

A: methane reacts with HBr/H₂O and O₂ to form alkyl bromides over the catalyst A;

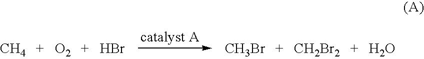

B: alkyl bromides are converted into hydrocarbons and HBr by the catalyst B.

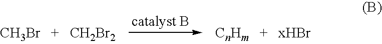

wherein, n=an integer which is equal to or greater than 2.

HBr generated in the reaction B can be reused in the reaction A to complete one cycle.

According to the present invention, the alkyl bromides formed in the step A can be all the same (e.g., 100% bromomethane) or, more typically, different (e.g., mixtures of bromomethane, dibromomethane, tribromomethane, etc). While in some circumstances it may be desirable to have bromomethane formed during the step A as a predominant product for the subsequent formation of higher hydrocarbons, the present system allows all types of methane bromides to be converted into higher hydrocarbons during step B. For certain product selectivities, polybromomethane may be desirable.

Some carbon oxides (e.g., CO and CO₂) are also formed as by-products during the process of oxidative bromination of methane (i.e., step A). The presence of these by-products will not influence subsequent operations, but such by-product formation decreases the yield of desired products. Careful selection of the concentration of O₂ in the feedstock to reaction step A and step A reaction conditions can minimize the formation of carbon oxides during the reaction.

In some embodiments, the first catalyst (catalyst A) comprises at least one of metal, metal halides and metal oxides supported on silicon dioxide, said metal is selected from alkaline earth metal, transition metal and/or lanthanide metal. According to some nonlimiting examples of the current invention, the metal is selected from the group consisting of Ru, Rh, Pd, Ir, Pt, Mg, Ca, Ba, Y, La, Sm, Bi, Fe, Co, Ni, Cu, V and Mo.

In some particular embodiments, the first catalyst (catalyst A) comprises at least one of metal and metal chlorides supported on silicon dioxide, said metal is selected from the group of transition metals. According to some nonlimiting examples of the current invention, the metal is selected from the group consisting of Ru, Rh, Pd, Ir and Pt, being preferred.

In other embodiments, the first catalyst (catalyst A) comprises at least one of metal oxides supported on silicon dioxide, the metal being selected from alkaline earth metals, transition metals and/or lanthanide metals. According to some nonlimiting examples of the current invention, the metal is selected from the group consisting of Mg, Ca, Ba, Y, La, Sm, Bi, Fe, Co, Ni, Cu, V and Mo, being preferred.

The first catalyst (catalyst A) can be prepared from a first catalyst precursor, wherein the first catalyst precursor comprises, but is not limited to, silicon dioxide, at least one of halides of metal selected from the group consisting of Ru, Rh, Pd, Ir and Pt, and/or at least one of nitrates, sulphates, halides, carbonates, oxalates or acetates of a metal selected from the group consisting of Mg, Ca, Ba, Y, La, Sm, Bi, Fe, Co, Ni, Cu, V and Mo.

In some additional embodiments, the first catalyst precursor comprises silicon dioxide, RuCl₃, and at least one of nitrates of metal selected from the group consisting of Mg, Ca, Ba, Y, La, Sm, Bi, Fe, Co, Ni, Cu, V and Mo, being preferred.

In some embodiments, the step A is carried out at a temperature between about 400° C. and about 800° C., and at a pressure between about 0.5 atm and about 10.0 atm. In certain nonlimiting examples, the step A is carried out at a temperature between about 500° C. and about 700° C., being preferred, and at a temperature of about 580° C. and about 660° C., being most preferred.

In some embodiments, methane monobromide separated from the products formed in the first reactor (step A) is directed into the second reactor (step B) for the synthesis of higher hydrocarbons. The methane monobromide may be separated from the product of step A exiting the first reactor by distillation and/or selective evaporation, for example, based upon the difference in boiling points of methane monobromide and the remaining reaction products of step A.

In some other embodiments, methane bromides separated from the product mixture of step A formed in the first reactor is directed into the second reactor for the synthesis of higher hydrocarbons. The presence of large concentrations of methane polybromides species in the feed to the second reactor (step B) can cause an increase in coke formation, which may partially or wholly deactivate the catalyst B. In many cases, it is desirable to feed only methane monobromide to the second reactor to improve the conversion efficiency to higher hydrocarbon compounds in step B. Careful selection of the ratio of HBr to methane and reaction conditions can considerably decrease the concentration of methane polybromides in the mixtures formed in the first reactor (step A). Further separating operations can be added between the two reactors (i.e., after step A and before step B) for separating desirable and undesirable methane bromides from the product mixture formed in step A and exiting the first reactor.

In certain embodiments, the product mixtures from step A without separation are directed into the second reactor (step B) for the synthesis of higher hydrocarbons.

The methane bromides produced from methane and HBr/H₂O and O₂ react over a second catalyst (catalyst B) to produce higher hydrocarbons and hydrogen bromide (i.e., step B). Optionally, the hydrogen bromide may be separated from the product mixture of step B (exiting the second reactor) and introduced into first reactor for re-use. In some embodiments, catalyst B comprises at least one metal oxide supported on HZSM-5, said metal is selected from alkaline earth metal, transition metal and/or lanthanide metal. In some embodiments, said metal is selected from the group consisting of Zn, Mg, Co, Cr, Cu, Ca, Fe, Ag, Pb, Bi, Ce, Sr, La, Y, Mn, Nb, Ti and mixtures thereof, being preferred. In some most preferred embodiments, said metal oxide is selected from MgO and ZnO.

Catalyst B may be prepared from a second catalyst precursor, wherein said second catalyst precursor comprises HZSM-5, and at least one of nitrates, sulphates, halides, carbonates, oxalates and acetates of metal selected from the group consisting of Zn, Mg, Co, Cr, Cu, Ca, Fe, Ag, Pb, Bi, Ce, Sr, La, Y, Mn, Nb and Ti. In some preferred embodiments, the second catalyst precursor comprises HZSM-5, and at least one of nitrates of metal selected from the group consisting of Zn, Mg, Cr, Ca, Fe, Ag, Pb, Bi, Ce, Sr, La and Y, and/or at least one of chlorides of metal selected from the group consisting of Co, Cu, Mn, Nb and Ti, being preferred.

In some embodiments, step B is carried out at a temperature between about 150° C. and about 500° C. According to some nonlimiting examples of the current invention, the step B is carried out at a temperature between about 200° C. and about 240° C., being preferred. In some embodiments, the step B is carried out at a pressure between about 0.5 atm and about 50.0 atm.

In most cases, the conversion of bromomethane to higher hydrocarbons is more than 50%. When certain catalyst is provided the conversion of bromomethane to higher hydrocarbon can be more than 90%. In general, a mixture of hydrocarbons is obtained, but careful selection of the metal-containing catalyst and reaction conditions can allow a tailored approach to hydrocarbon product formation. In some embodiments, the proportion of C₃-C₈ hydrocarbons in the products is more than 80%. In some preferred embodiments, the proportion of C₃-C₈ hydrocarbons in the products is more than 95%, and the proportion of aromatics in the products is less than 5%.

Embodiments of the inventive process have wide applications in preparing chemicals. Embodiments of the present invention process are energy-saving. For example, when gasoline is prepared by the inventive process, the two exothermic reactions, steps A and B, can be carried out under atmospheric pressure. In embodiments of the inventive process, the raw materials for preparing alkyl bromides are O₂, natural gas and HBr/H₂O, in which HBr/H₂O solution is used as bromine source instead of Br₂. The use of HBr/H₂O offers a much safer solution than processes utilizing Br₂ because the reactions are strongly exothermic, and H₂O in HBr/H₂O absorb and mitigate the generated heat. Thus, the temperature of the catalytic bed can be controlled. Furthermore, in some embodiments of the present invention, HBr is regenerated in the process of converting alkyl bromides into higher hydrocarbons and is re-used in the step A of the invention. In addition, the embodiments of the present invention do not require a separate step to regenerate Br₂.

### EXAMPLES

The following examples are provided to further illustrate the invention. They represent specific embodiments of the current invention and should not be interpreted or construed as limitations to the scope of the invention.

### Example 1-23

### Oxidative Bromination of Alkanes

The catalysts were prepared as follows: Silica (10 g, S_{BET}=1.70 m²/g), RuCl₃ solution (0.00080 g Ru/mL) and corresponding metal nitrates solution (0.10 M) were mixed in a mole ratio of components of catalysts given in Table 1, stirred at ambient temperature for 0.5 h, dried at 110° C. for 4 h, and then calcined at 450° C. for 12 h.

The catalytic reaction was carried out in the quartz-tube reactor (i.d. 0.80 cm, length 60 cm) at the temperatures shown in Table 1, packed with 1.0000 g catalyst with both ends filled with quartz sand, with reactant flows: 5.0 mL/min of methane, 5.0 mL/min of oxygen, 4.0 mL (liquid)/h of 40 wt % HBr/H₂O solution. The products were analyzed by gas phase chromatography. Results are set forth in Table 1.

TABLE 1
Components of Catalysts, Temperature and Results of the Reaction

| | Temperature | | Conversion | Selectivity (mol %) | | | |
|---|---|---|---|---|---|---|---|
| Sample | (° C.) | Catalysts | (mol %) | CH₃Br | CH₂Br₂ | CO | CO₂ |
| 1 | 580 | 0.1% Ru/SiO₂ | 38.4 | 52.9 | 0 | 47.1 | 0 |
| 2 | 580 | 0.1% Rh/SiO₂ | 35.9 | 37.9 | 0 | 62.1 | 0 |
| 3 | 580 | 5% Mg 0.1% Ru/SiO₂ | 32.1 | 53.1 | 4.5 | 42.4 | 0 |
| 4 | 580 | 5% Ca 0.1% Ru/SiO₂ | 20.9 | 33.1 | 3.3 | 63.6 | 0 |
| 5 | 580 | 5% Ba 0.1% Ru/SiO₂ | 25.9 | 76.8 | 6.6 | 16.6 | 0 |
| 6 | 580 | 5% Y 0.1% Ru/SiO₂ | 69.9 | 15.4 | 1.8 | 77.7 | 5.1 |
| 7 | 580 | 5% La 0.1% Ru/SiO₂ | 72.2 | 30.7 | 5.6 | 61.0 | 2.7 |
| 8 | 580 | 5% Sm 0.1% Ru/SiO₂ | 81.4 | 7.6 | 2.1 | 86.9 | 3.4 |
| 9 | 600 | 5% Sm 0.1% Ru/SiO₂ | 86.6 | 6.8 | 1.2 | 88.0 | 4.0 |
| 10 | 580 | 2.5% Ba 2.5% La 0.1% Ru/SiO₂ | 42.9 | 55.9 | 6.1 | 38.0 | 0 |
| 11 | 580 | 2.5% Ba 2.5% La/SiO₂ | 15.7 | 52.2 | 14.6 | 33.2 | 0 |
| 12 | 600 | 2.5% Ba 2.5% La 0.1% Ru/SiO₂ | 58.8 | 53.4 | 4.9 | 41.7 | 0 |
| 13 | 580 | 2.5% Ba 2.5% Sm 0.1% Ru/SiO₂ | 34.5 | 61.8 | 9.1 | 29.1 | 0 |
| 14 | 600 | 2.5% Ba 2.5% Sm 0.1% Ru/SiO₂ | 41.5 | 57.2 | 5.0 | 37.8 | 0 |
| 15 | 580 | 2.5% Ba 2.5% Bi 0.1% Ru/SiO₂ | 18.2 | 60.2 | 16.2 | 23.6 | 0 |
| 16 | 600 | 2.5% Ba 2.5% Bi 0.1% Ru/SiO₂ | 37.1 | 49.9 | 5.8 | 44.3 | 0 |
| 17 | 600 | 2.5% Ba 2.5% La 0.5% Bi 0.1% Ru/SiO₂ | 50.0 | 54.4 | 7.0 | 38.6 | 0 |
| 18 | 600 | 2.5% Ba 2.5% La 0.5% Fe 0.1% Ru/SiO₂ | 59.3 | 51.7 | 3.1 | 40.4 | 4.8 |
| 19 | 600 | 2.5% Ba 2.5% La 0.5% Co 0.1% Ru/SiO₂ | 52.1 | 52.2 | 3.4 | 38.2 | 6.2 |
| 20 | 600 | 2.5% Ba 2.5% La 0.5% Ni 0.1% Ru/SiO₂ | 62.9 | 54.5 | 5.3 | 34.6 | 5.6 |
| 21 | 600 | 2.5% Ba 2.5% La 0.5% Cu 0.1% Ru/SiO₂ | 41.3 | 51.4 | 2.8 | 39.4 | 6.4 |
| 22 | 600 | 2.5% Ba 2.5% La 0.5% V 0.1% Ru/SiO₂ | 57.6 | 50.5 | 3.0 | 38.0 | 8.5 |
| 23 | 600 | 2.5% Ba 2.5% La 0.5% Mo 0.1% Ru/SiO₂ | 53.6 | 52.1 | 2.4 | 36.0 | 9.5 |

Notes:
methane: 5.0 mL/min, oxygen: 5.0 mL/min, 40 wt % HBr/H2O: 4.0 mL (liquid)/h, catalyst: 1.0000 g

### Example 24

The catalysts were prepared as follows: Silica (10 g, S_{BET}=0.50 m²/g), RuCl₃ solution (0.00080 g Ru/mL), La(NO₃)₃ solution (0.10 M), Ba(NO₃)₂ solution (0.10 M), Ni(NO₃)₂ solution (0.10 M) were mixed in a mole ratio of 2.5% La, 2.5% Ba, 0.5% Ni, 0.1% Ru and 94.4% SiO₂. The mixture was stirred at ambient temperature for 0.5 h, dried at 110° C. for 4 h, and then calcined at 450° C. for 12 h to give the catalyst with composition as La2.5% Ba2.5% Ni0.5% Ru0.1%/SiO₂.

The catalytic reaction was carried out in the quartz-tube reactor (i.d. 1.50 cm, length 60 cm) at 660° C., packed with 5.000 g catalyst with both ends filled with quartz sand, with reactant flows: 15.0 mL/min of methane, 5.0 mL/min of oxygen, 6.0 mL (liquid)/h of 40 wt % HBr/H₂O solution. The products were analyzed by a gas phase chromatography. Methane conversion was 32.0%, and the selectivities of CH₃Br, CH₂Br₂, CO and CO₂ were 80.8%, 0.67%, 15.7% and 2.9%, respectively.

### Example 25-38

### Conversion from Alkane Bromide to Higher Hydrocarbons

Preparation of Catalyst ZnO/HZSM-5 and MgO/HZSM-5

The catalysts C₁-C₁₄ of example 25-38 in Table 2 were prepared as follows: HZSM-5 (Si/Al=360, 283 m²/g), water and Zn(NO₃)₂.6H₂O (or Mg(NO₃)₂₋₆H₂O) were mixed in a ratio given in Table 2 and stirred, impregnated at ambient temperature for 12 h, dried at 120° C. for 4 h, and then calcined at 450° C. for 8 h. The catalyst was tabletted at 100 atm and lastly crushed and sieved to 40-60 mesh to afford the catalysts shown in Table 2.

TABLE 2

| Sample | Catalyst | Component | HZSM-5 (g) | H₂O (mL) | Mg(NO₃)₂•6H₂O (g) | Zn(NO₃)₂•6H₂O (g) |
|---|---|---|---|---|---|---|
| 25 | C1 | 5.0 wt % ZnO/HZSM-5 | 10.0000 | 30.0 | 0 | 1.8276 |
| 26 | C2 | 6.0 wt % ZnO/HZSM-5 | 10.0000 | 30.0 | 0 | 2.1931 |
| 27 | C3 | 8.0 wt % ZnO/HZSM-5 | 10.0000 | 30.0 | 0 | 2.9242 |
| 28 | C4 | 10.0 wt % ZnO/HZSM-5 | 10.0000 | 30.0 | 0 | 3.6522 |
| 29 | C5 | 12.0 wt % ZnO/HZSM-5 | 10.0000 | 30.0 | 0 | 4.3862 |
| 30 | C6 | 14.0 wt % ZnO/HZSM-5 | 10.0000 | 30.0 | 0 | 5.1173 |
| 31 | C7 | 15.0 wt % ZnO/HZSM-5 | 10.0000 | 30.0 | 0 | 5.4828 |
| 32 | C8 | 5.0 wt % MgO/HZSM-5 | 10.0000 | 30.0 | 3.2051 | 0 |
| 33 | C9 | 6.0 wt % MgO/HZSM-5 | 10.0000 | 30.0 | 3.2051 | 0 |
| 34 | C10 | 8.0 wt % MgO/HZSM-5 | 10.0000 | 30.0 | 5.1281 | 0 |
| 35 | C11 | 10.0 wt % MgO/HZSM-5 | 10.0000 | 30.0 | 6.4102 | 0 |
| 36 | C12 | 12.0 wt % MgO/HZSM-5 | 10.0000 | 30.0 | 7.6922 | 0 |
| 37 | C13 | 14.0 wt % MgO/HZSM-5 | 10.0000 | 30.0 | 8.9743 | 0 |
| 38 | C14 | 15.0 wt % MgO/HZSM-5 | 10.0000 | 30.0 | 9.6153 | 0 |

The catalysts of example 25-38 were used to convert CH₃Br into higher hydrocarbons. The reaction was carried out in the glass-tube reactor (i.d. 1.50 cm) with 8.0 g catalyst at 240° C., with a flow of 6.8 mL/min of CH₃Br. The products were analyzed by gas phase chromatography. The conversion of CH₃Br and the selectivities of higher hydrocarbons are set forth in Table 3. Cₙ in Table 3 means the total amount of alkanes containing n carbons.

TABLE 3
Conversion Rate of CH₃Br and Product Selectivity

| | | Alkanes and Alkenes | | | | | | | | Aromatics | | | | | | |
|---|---|---|---|---|---|---|---|---|---|---|---|---|---|---|---|---|
| | X | C₂ | C₃ | C₄ | C₅ | C₆ | C₇ | C₈ | C₉ | C₇ | C₈ | C₉ | C₁₀ | C₁₁ | C₁₂ | C₁₃ |
| Catalyst | (%) | (%) | (%) | (%) | (%) | (%) | (%) | (%) | (%) | (%) | (%) | (%) | (%) | (%) | (%) | (%) |
| C1 | 91.0 | 2.8 | 15.3 | 44.2 | 20.9 | 9.7 | 3.4 | 0.0 | 0.2 | 0.1 | 0.5 | 1.6 | 0.7 | 0.2 | 0.3 | 0.1 |
| C2 | 97.4 | 1.6 | 12.2 | 44.0 | 21.6 | 10.4 | 3.8 | 0.7 | 0.3 | 0.1 | 1.0 | 2.6 | 1.0 | 0.3 | 0.3 | 0.1 |
| C3 | 98.3 | 1.6 | 13.7 | 42.2 | 18.9 | 9.3 | 4.8 | 1.2 | 0.3 | 0.1 | 1.3 | 4.0 | 1.5 | 0.4 | 0.6 | 0.1 |
| C4 | 98.7 | 1.6 | 9.1 | 33.0 | 22.2 | 19.0 | 4.3 | 1.2 | 0.4 | 0.2 | 1.4 | 4.3 | 1.8 | 0.5 | 0.8 | 0.2 |
| C5 | 95.4 | 1.9 | 12.0 | 42.4 | 21.4 | 12.7 | 3.1 | 0.3 | 0.1 | 0.0 | 0.3 | 1.1 | 4.4 | 0.1 | 0.2 | 0.0 |
| C6 | 94.4 | 1.9 | 15.5 | 47.6 | 19.4 | 7.6 | 2.7 | 0.6 | 0.2 | 0.1 | 0.7 | 2.2 | 0.9 | 0.2 | 0.3 | 0.1 |
| C7 | 92.0 | 1.8 | 14.9 | 44.7 | 20.9 | 10.9 | 4.4 | 0.3 | 0.1 | 0.0 | 0.3 | 1.0 | 0.4 | 0.1 | 0.2 | 0.0 |
| C8 | 99.6 | 1.9 | 10.9 | 45.9 | 20.5 | 11.1 | 3.6 | 0.7 | 0.5 | 0.3 | 1.1 | 0.5 | 0.8 | 1.2 | 0.4 | 0.6 |
| C9 | 99.6 | 2.6 | 9.4 | 44.3 | 22.4 | 12.5 | 5.5 | 0.7 | 0.4 | 0.0 | 0.7 | 0.3 | 0.3 | 0.5 | 0.2 | 0.2 |
| C10 | 99.6 | 3.3 | 5.7 | 49.2 | 27.9 | 4.7 | 6.3 | 0.6 | 0.4 | 0.0 | 0.6 | 0.2 | 0.6 | 0.3 | 0.1 | 0.1 |
| C11 | 99.6 | 2.9 | 7.5 | 44.6 | 22.8 | 10.5 | 4.3 | 0.9 | 0.5 | 0.3 | 1.9 | 0.8 | 0.9 | 1.3 | 0.5 | 0.3 |
| C12 | 99.3 | 2.5 | 8.5 | 39.6 | 24.7 | 12.0 | 5.9 | 1.1 | 0.5 | 0.0 | 1.5 | 0.6 | 1.7 | 0.8 | 0.5 | 0.1 |
| C13 | 99.6 | 3.3 | 5.7 | 49.1 | 26.7 | 4.1 | 6.3 | 0.9 | 0.5 | 0.0 | 0.9 | 0.4 | 0.7 | 0.7 | 0.2 | 0.5 |
| C14 | 99.5 | 2.0 | 6.9 | 46.5 | 25.5 | 10.0 | 4.2 | 0.9 | 0.5 | 0.2 | 1.0 | 0.4 | 0.6 | 0.7 | 0.5 | 0.1 |

Note:
X means the conversion rate of CH₃Br.

### Example 39-53

The catalysts C15-C29 of example 39-53 in Table 4 were prepared as follows: HZSM-5 (Si/Al=360, 283 m²/g), water and corresponding salts were mixed in a ratio given in table 4 and stirred, impregnated at ambient temperature for 12 h, dried at 120° C. for 4 h, and then calcined at 450° C. for 8 h. The catalyst was tabletted at 100 atm and then crushed and sieved to 40-60 mesh to afford the catalysts shown in Table 4.

TABLE 4

| | | | | | Second | | |
|---|---|---|---|---|---|---|---|
| Sample | Catalyst | Catalyst | First composition | | composition | | HZSM-5 (g) |
| 39 | C15 | Co/HZSM-5 | CoCl₂•6H₂O | 1.5877 g | H₂O | 30 mL | 10.000 |
| 40 | C16 | Cr/HZSM-5 | Cr(NO₃)₃•9H₂O | 1.3160 g | H₂O | 30 mL | 10.000 |
| 41 | C17 | Cu/HZSM-5 | CuCl₂•2H₂O | 1.0722 g | H₂O | 30 mL | 10.000 |
| 42 | C18 | Ca/HZSM-5 | Ca(NO₃)₂•4H₂O | 2.1085 g | H₂O | 30 mL | 10.000 |
| 43 | C19 | Fe/HZSM-5 | Fe(NO₃)₃•9H₂O | 2.5250 g | H₂O | 30 ml | 10.000 |
| 44 | C20 | Ag/HZSM-5 | AgNO₃ | 0.7322 g | H₂O | 30 ml | 10.000 |
| 45 | C21 | Pb/HZSM-5 | Pb(NO₃)₂ | 0.7426 g | H₂O | 30 ml | 10.000 |
| 46 | C22 | Bi/HZSM-5 | Bi(NO₃)₃•5H₂O | 1.0413 g | H₂O | 30 ml | 10.000 |
| 47 | C23 | Ce/HZSM-5 | Ce(NO₃)₂•6H₂O | 1.3229 g | H₂O | 30 ml | 10.000 |
| 48 | C24 | Sr/HZSM-5 | Sr(NO₃)₂ | 1.0212 g | H₂O | 30 ml | 10.000 |
| 49 | C25 | La/HZSM-5 | La(NO₃)₃•6H₂O | 1.3291 g | H₂O | 30 ml | 10.000 |
| 50 | C26 | Y/HZSM-5 | Y(NO₃)₃•6H₂O | 1.6963 g | H₂O | 30 ml | 10.000 |
| 51 | C27 | Mn/HZSM-5 | MnCl₂ | 1.3800 g | H₂O | 30 ml | 10.000 |
| 52 | C28 | Nb/HZSM-5 | NbCl₅ | 1.0514 g | C₂H₅OH | 40 ml | 10.000 |
| 53 | C29 | Ti/HZSM-5 | TiCl₄ | 1.000 ml | C₂H₅OH | 40 ml | 10.000 |

The catalysts of example 39-53 were used to convert CH₃Br into higher hydrocarbons. The reaction was carried out in the glass-tube reactor (i.d. 1.50 cm) with 8.0 g catalyst at 200-240° C., with a flow of 6.8 mL/min of CH₃Br. The products were analyzed by gas phase chromatography. The conversion of CH₃Br and the selectivity of higher hydrocarbons are given in Table 5. Cₙ in Table 5 means the total amount of alkanes containing n carbons.

TABLE 5
Conversion Rate of CH₃Br and Product Selectivity

| | | | X | | | | | | |
|---|---|---|---|---|---|---|---|---|---|
| Catalyst | Catalyst | T (° C.) | (%) | C₂ (%) | C₃ (%) | C₄ (%) | C₅ (%) | C₆ (%) | C₇ (%) |
| C15 | Co/HZSM-5 | 240 | 84.9 | 4.7 | 10.8 | 32.6 | 18.1 | 17.2 | 16.6 |
| C16 | Cr/HZSM-5 | 200 | 44.0 | 0 | 13.6 | 73.8 | 12.6 | 0 | 0 |
| C16 | Cr/HZSM-5 | 220 | 79.8 | 6.8 | 15.6 | 45.2 | 14.6 | 8.5 | 9.4 |
| C16 | Cr/HZSM-5 | 240 | 81.1 | 9.3 | 16.9 | 36.1 | 22.9 | 8.6 | 6.2 |
| C17 | Cu/HZSM-5 | 200 | 62.7 | 0 | 11.6 | 52.7 | 22.2 | 13.4 | 0 |
| C17 | Cu/HZSM-5 | 220 | 67.5 | 4.4 | 25.2 | 45.8 | 16.6 | 4.5 | 3.5 |
| C17 | Cu/HZSM-5 | 240 | 71.1 | 1.8 | 7.0 | 22.1 | 60.3 | 4.2 | 4.6 |
| C18 | Ca/HZSM-5 | 220 | 94.8 | 0 | 13.8 | 44.4 | 15.3 | 17.1 | 9.4 |
| C18 | Ca/HZSM-5 | 240 | 95.0 | 0 | 21.3 | 49.5 | 17.6 | 6.8 | 4.9 |
| C19 | Fe/HZSM-5 | 200 | 39.7 | 8.2 | 8.6 | 41.1 | 18.4 | 16.7 | 7.0 |
| C19 | Fe/HZSM-5 | 220 | 75.6 | 12.0 | 20.2 | 45.0 | 10.1 | 12.7 | 0 |
| C19 | Fe/HZSM-5 | 240 | 69.6 | 25.9 | 20.8 | 32.2 | 11.3 | 4.8 | 5.0 |
| C20 | Ag/HZSM-5 | 200 | 24.6 | 0 | 10.9 | 29.2 | 27.1 | 15.3 | 17.4 |
| C20 | Ag/HZSM-5 | 220 | 50.9 | 25.9 | 20.8 | 32.2 | 11.3 | 4.8 | 5.0 |
| C20 | Ag/HZSM-5 | 240 | 70.0 | 0 | 14.7 | 56.8 | 22.4 | 2.5 | 3.7 |
| C21 | Pb/HZSM-5 | 220 | 70.1 | 25.9 | 20.7 | 32.2 | 11.2 | 4.9 | 5.1 |
| C21 | Pb/HZSM-5 | 240 | 82.6 | 7.7 | 14.9 | 32.3 | 19.5 | 12.6 | 13.5 |
| C22 | Bi/HZSM-5 | 200 | 33.8 | 6.1 | 7.1 | 30.3 | 23.2 | 30.6 | 2.6 |
| C23 | Ce/HZSM-5 | 200 | 70.6 | 2.9 | 4.2 | 22.9 | 25.8 | 14.5 | 29.6 |
| C23 | Ce/HZSM-5 | 220 | 76.3 | 0 | 10.9 | 29.2 | 27.1 | 15.3 | 17.4 |
| C23 | Ce/HZSM-5 | 240 | 77.0 | 25.9 | 20.8 | 32.2 | 11.3 | 4.8 | 5.0 |
| C24 | Sr/HZSM-5 | 200 | 62.5 | 11.2 | 4.4 | 36.7 | 39.2 | 1.3 | 7.0 |
| C24 | Sr/HZSM-5 | 220 | 85.9 | 6.8 | 15.6 | 45.2 | 14.6 | 8.5 | 9.4 |
| C24 | Sr/HZSM-5 | 240 | 98.1 | 9.3 | 16.9 | 36.1 | 22.9 | 8.6 | 6.2 |
| C25 | La/HZSM-5 | 200 | 63.7 | 2.9 | 4.2 | 22.9 | 25.8 | 14.5 | 29.6 |
| C25 | La/HZSM-5 | 220 | 70.8 | 0 | 10.9 | 29.2 | 27.1 | 15.3 | 17.4 |
| C25 | La/HZSM-5 | 240 | 75.8 | 25.9 | 20.8 | 32.2 | 11.3 | 4.8 | 5.0 |
| C26 | Y/HZSM-5 | 200 | 13.3 | 0 | 6.7 | 36.6 | 29.1 | 18.3 | 9.2 |
| C26 | Y/HZSM-5 | 220 | 64.2 | 3.8 | 23.5 | 39.8 | 19.7 | 9.8 | 3.3 |
| C26 | Y/HZSM-5 | 240 | 69.2 | 5.4 | 11.9 | 42.5 | 24.4 | 10.6 | 5.1 |
| C27 | Mn/HZSM-5 | 200 | 67.0 | 7.1 | 14.0 | 39.4 | 24.5 | 10.3 | 4.6 |
| C27 | Mn/HZSM-5 | 240 | 83.7 | 3.4 | 6.5 | 37.9 | 26.4 | 13.0 | 12.7 |
| C28 | Nb/HZSM-5 | 200 | 68.5 | 3.2 | 17.1 | 40.5 | 22.1 | 10.4 | 6.5 |
| C28 | Nb/HZSM-5 | 240 | 68.5 | 3.6 | 5.9 | 30.9 | 23.0 | 15.2 | 21.4 |
| C29 | Ti/HZSM-5 | 220 | 46.8 | 4.2 | 13.1 | 41.7 | 23.9 | 10.5 | 6.7 |
| C29 | Ti/HZSM-5 | 240 | 79.2 | 4.9 | 22.1 | 41.6 | 19.4 | 5.6 | 6.5 |

The Reaction-in-series of Oxidative Bromination of Methane and Producing Higher Hydrocarbon from CH₃Br

### Example 54

For preparing the catalyst, Silica (10 g, S_{BET}=^{0.50} m²/g), RuCl₃ solution (0.00080 g Ru/mL), La(NO₃)₃ solution (0.10 M), Ba(NO₃)₂ solution (0.10 M), Ni(NO₃)₂ solution (0.10 M) were mixed in a mole ratio of 2.5% La, 2.5% Ba, 0.5% Ni, 0.1% Ru and 94.4% SiO₂. The result solution was stirred at ambient temperature for 0.5 h, dried at 110° C. for 4 h, and then calcined at 450° C. for 12 h to give the catalyst with component as La2.5% Ba2.5% Ni0.5% Ru0.1%/SiO₂.

The catalytic reaction was carried out in the quartz-tube reactor (i.d. 1.50 cm, length 60 cm) at 660° C., packed with 5.000 g catalyst with both ends filled with quartz sand, with reactant flows: 15.0 mL/min of methane, 5.0 mL/min of oxygen, 6.0 mL (liquid)/h of 40 wt % HBr/H₂O solution. The products were analyzed by gas phase chromatography. Methane conversion was 32.0%, and the selectivities of CH₃Br, CH₂Br₂, CO and CO₂ were 80.8%, 0.67%, 15.7% and 2.9%, respectively. The composite undergone first step reaction was directly introduced into glass-tube reactor (i.d. 1.5 cm) at 240° C., which was packed with 8.0 g 14.0 wt % MgO/HZSM-5 catalyst. The final products were analyzed by gas phase chromatography. The conversions of CH₃Br and CH₂Br₂ were about 100% through the second reactor and the products were hydrocarbons of C₂˜C₁₃. The similar result was achieved using 8.0 g 14.0 wt % ZnO/HZSM-5 as a substitute for the former catalyst in the second reactor.

### Methane Flow Rate Changes to Affect Conversion and Product Selectivities in Step A

### Example 55

In another example, catalytic reaction was also carried out in the quartz-tube reactor (i.d. 1.50 cm, length 60 cm) at 660° C., packed with 5.000 g catalyst, but with reactant flows: 20.0 mL/min of methane, 5.0 mL/min of oxygen, 6.0 mL (liquid)/h of 40 wt % HBr/H₂O solution. The products were analyzed by gas phase chromatography. Methane conversion was 26.7%, and the selectivities of CH₃Br, CH₂Br₂, CO and CO₂ were 82.2%, 3.3%, 11.9% and 2.6%, respectively. The composite undergone first step reaction was directly introduced into glass-tube reactor (i.d. 1.5 cm) at 240° C., which was packed with 8.0 g 14.0 wt % MgO/HZSM-5 catalyst. The final products were analyzed by gas phase chromatography. The conversions of CH₃Br and CH₂Br₂ were about 100% through the second reactor and the products were hydrocarbons of C₂˜ C₁₃.

### Example 56

CO is the main by-product in the first step reaction and it is difficult to separate from CH₄. So CO and CH₄ were returned into first reactor for further reaction without separation. CH₄, O₂, CO(N2 as internal standard) and 40 wt % HBr/H₂O (6.0 mL/h) were fed together into the first reactor, with flows: 15.0 mL/min of CH₄, 5.0 mL/min of O₂, 3.0 mL/min of CO, 5.0 mL/min of N₂, 6.0 mL/h of 40 wt % HBr/H₂O (liquid). The reaction was carried out at 660° C. and the conversion of methane was 30.4%, the selectivities of CH₃Br, CH₃Br₂ and CO₂ were 86.5%, 1.7% and 11.8%, respectively. The total selectivity of CH₃Br and CH₃Br₂ was 88.2%. The composite through the first reaction was directly introduced into the second reactor in which CH₃Br and CH₃Br₂ were all converted into hydrocarbons of C₂˜ C₁₃.

What the claim is:

1. A process for converting methane into higher hydrocarbons comprising the steps of:
(a) contacting methane with a source of oxygen and hydrogen bromide to form one or more methane bromide compounds, in the presence of a first catalyst within a first reactor;
(b) converting the methane bromides into C₃˜C₁₃ hydrocarbons and hydrogen bromide in the presence of a second catalyst within a second reactor.

2. The process of claim 1, further comprises the step of (c) recovering the hydrogen bromide produced in step (b) and recycling the recovered hydrogen bromide into step (a).

3. The process according to claim 1, wherein the first catalyst comprises at least one of metal, metal halides and metal oxides supported on silicon dioxide, wherein said metal is selected from the group consisting of Ru, Rh, Pd, Ir, Pt, Mg, Ca, Ba, Y, La, Sm, Bi, Fe, Co, Ni, Cu, V and Mo.

4. The process according to claim 3, wherein the first catalyst comprises at least one of metal and metal chlorides supported on silicon dioxide, wherein said metal is selected from the group consisting of Ru, Rh, Pd, Ir and Pt.

5. The process according to claim 3, wherein the first catalyst comprises at least one metal oxide supported on silicon dioxide, wherein said metal is selected from the group consisting of Mg, Ca, Ba, Y, La, Sm, Bi, Fe, Co, Ni, Cu, V and Mo.

6. The process according to claim 3, wherein the first catalyst is prepared from a first catalyst precursor, wherein said first catalyst precursor comprises silicon dioxide, at least one halide of one or more metals selected from the group of Ru, Rh, Pd, Ir and Pt, and/or at least one of nitrates, sulphates, halides, carbonates, oxalates or acetates of one or more metals selected from the group consisting of Mg, Ca, Ba, Y, La, Sm, Bi, Fe, Co, Ni, Cu, V and Mo.

7. The process according to claim 6, wherein the first catalyst precursor comprises silicon dioxide, RuCl₃, and at least one nitrate of one or more metals selected from the group consisting of Mg, Ca, Ba, Y, La, Sm, Bi, Fe, Co, Ni, Cu, V and Mo.

8. The process according to claim 1, wherein step (a) is carried out at a temperature between about 400° C. and about 800° C.

9. The process according to claim 1, wherein the step (a) is carried out at a pressure between about 0.5 atm and about 10.0 atm.

10. The process of claim 1, wherein the second catalyst comprises at least one metal oxide supported on HZSM-5, wherein said metal is selected from the group consisting of Zn, Mg, Co, Cr, Cu, Ca, Fe, Ag, Pb, Bi, Ce, Sr, La, Y, Mn, Nb, Ti and mixtures thereof.

11. The process according to claim 1, wherein the second catalyst is prepared from a second catalyst precursor, wherein said second catalyst precursor comprises HZSM-5, and at least one of nitrates, sulphates, halides, carbonates, oxalates and acetates of one or more metals selected from the group consisting of Zn, Mg, Co, Cr, Cu, Ca, Fe, Ag, Pb, Bi, Ce, Sr, La, Y, Mn, Nb and Ti.

12. The process according to claim 11, wherein the second catalyst precursor comprises HZSM-5, and at least one nitrate of one or more metals selected from the group consisting of Zn, Mg, Cr, Ca, Fe, Ag, Pb, Bi, Ce, Sr, La and Y, and/or at least one of chlorides of metal selected from the group consisting of Co, Cu, Mn, Nb and Ti.

13. The process according to claim 1, wherein the step (b) is carried out at a temperature between about 150° C. and about 500° C.

14. The process according to claim 1, wherein the step (b) is carried out at a pressure between about 0.5 atm and about 50.0 atm.
